(19) United States
(12) Patent Application Publication
(10) Pub. No.: US 2026/0345132 A1
(43) Pub. Date: Nov. 5, 2026

(54) SAMPLE COLLECTION DEVICE

(71) Applicant: Siro Diagnostics, Inc., San Diego, CA (US)

(72) Inventors: Clint Sever, San Diego, CA (US); Joseph D. Kowalski, San Diego, CA (US); Jeremy D. Shaver, San Diego, CA (US); Scott Beach, San Diego, CA (US); Robert K. Shanahan, San Diego, CA (US); Steven M. Venz, San Diego, CA (US); Randy Murray, San Diego, CA (US)

(21) Appl. No.: 19/488,364

(22) PCT Filed: May 31, 2024
(86) PCT No.: PCT/US2024/031843

§ 371 Date: Nov. 26, 2025

Related U.S. Application Data
(60) Provisional application No. 63/470,267, filed on Jun. 1, 2023.

(51) Int. Cl. A61B 10/00

(52) U.S. Cl. CPC A61B 10/0045; A61B 2560/045

(57) ABSTRACT

Devices, systems, and methods for collecting samples for medical testing. A sample collection device includes a collection chamber coupled to a body portion. The collection chamber extends from a first collection chamber end to a second collection chamber end. The collection chamber includes an opening proximate the second collection chamber end. The opening is configured to receive a first volume of the biological sample. A projection extends into the collection chamber. The collection chamber is configured to translate relative to the projection between a first position and a second position, wherein translation of the collection chamber from the first position to the second position causes a second volume of the biological sample to be released from the collection chamber through the opening.

### CROSS-REFERENCE TO RELATED APPLICATIONS

This application claims the benefit of and priority to U.S. Provisional Patent Application No. 63/470,267, filed on Jun. 1, 2023, the entirety of which is incorporated by reference herein.

### TECHNOLOGICAL FIELD

The present technology relates generally to systems and methods of for collecting samples for testing.

### BACKGROUND

Sample collection devices may be configured to collect cells and other biological material including, for example, from urine, from blood, from plasma, from saliva, etc.

### SUMMARY

A set of embodiments relate to a sample collection device for collecting a biological sample for analysis in a detection system. The sample collection device includes a body portion extending along a body axis from a first body end to a second body end. The sample collection device includes collection chamber coupled to the body portion and extending from a first collection chamber end to a second collection chamber end. The collection chamber includes an opening proximate the second collection chamber end. The opening is configured to receive the biological sample. The sample collection device includes a wick positioned within the collection chamber. The wick is configured to receive the biological sample from the opening and store a first volume of the biological sample. The sample collection device includes a plunger extending from the body portion proximate the second body end such that a portion of the plunger is positioned within the collection chamber. The collection chamber is configured to translate relative to the plunger along the body axis between a first position and a second position. The translation of the collection chamber from the first position to the second position causes the second volume of the biological sample to be released from the wick and dispensed from the opening in the collection chamber.

In various embodiments, the first volume is greater than the second volume. The body portion may include a shoulder proximate the second body end, and the shoulder prevents the collection chamber from translating beyond the second position. The collection chamber may include a vent proximate the first collection chamber end, and the opening is in fluid communication with the vent while the collection chamber is in the first position and the opening is sealed off from the vent while the collection chamber is in the second position. The plunger may include a sealing member proximate an end of the plunger, the sealing member including an over-molded portion. The sample collection device may include a shroud coupled to the body portion such that the shroud encompasses at least a portion of the plunger. The shroud may include a projection configured to interface with the collection chamber to prevent the collection chamber from translating beyond the first position. The vent may be one of a plurality of vents equally spaced about an outer diameter of the collection chamber. The opening may be one of a plurality of openings equally spaced apart about the second collection chamber end. The sample collection device may include a shroud coupled to the body portion proximate the second body end, the shroud configured to receive a portion of the collection chamber and secure the collection chamber in the second position. The collection chamber may include a transparent portion such that the wick is visible via the transparent portion. The wick may be configured to reveal a visual indicator as the wick receives the biological sample saturates the wick.

Another set of embodiments relate to a system. The system includes a cartridge device including a cartridge opening. The system includes a sample collection device configured to collect a biological sample for analysis and eject the biological sample into the cartridge opening. The sample collection device includes a body portion extending along a body axis from a first body end to a second body end and a collection chamber configured to be inserted into the cartridge opening and coupled to the body portion and extending from a first collection chamber end to a second collection chamber end. The collection chamber includes an opening proximate the second collection chamber end, the opening configured to receive the biological sample. The body portion includes a wick positioned within the collection chamber, the wick configured to receive the biological sample from the opening and store a first volume of the biological sample. The body further includes a plunger extending from the body portion proximate the second body end such that a portion of the plunger is positioned within the collection chamber. The collection chamber is configured to translate relative to the plunger along the body axis between a first position and a second position in response to an axial force applied to the body portion while the collection chamber is positioned within the cartridge opening, and translation of the collection chamber from the first position to the second position causes a second volume of the biological sample to be released from the wick and dispensed into the cartridge opening.

In various embodiments, the first volume is greater than the second volume. The body portion may include a shoulder proximate the second body end, and the shoulder prevents the collection chamber from translating beyond the second position. The collection chamber may include a vent proximate the first collection chamber end, and the opening is in fluid communication with the vent while the collection chamber is in the first position and the opening is sealed off from the vent while the collection chamber is in the second position. The plunger may include a sealing member proximate an end of the plunger, the sealing member including an over-molded portion. The sample collection device may include a shroud coupled to the body portion such that the shroud encompasses at least a portion of the plunger. The shroud may include a projection configured to interface with the collection chamber to prevent the collection chamber from translating beyond the first position. The sample collection device may further include a shroud coupled to the body portion proximate the second body end, the shroud configured to receive a portion of the collection chamber and secure the collection chamber in the second position.

Another set of embodiments relate to a sample collection device for collecting a biological sample for analysis in a detection system. The sample collection device includes a collection chamber coupled to a body portion and extending from a first collection chamber end to a second collection chamber end. The collection chamber includes an opening proximate the second collection chamber end. The opening is configured to receive a first volume of the biological sample. The sample collection device includes a projection extending into the collection chamber. The collection chamber is configured to translate relative to the projection between a first position and a second position, and translation of the collection chamber from the first position to the second position causes a second volume of the biological sample to be released from the collection chamber through the opening.

According to various embodiments, the collection chamber includes a vent proximate the first collection chamber end, and the opening is in fluid communication with the vent while the collection chamber is in the first position and the opening is sealed off from the vent while the collection chamber is in the second position.

### BRIEF DESCRIPTION OF THE DRAWINGS

Example embodiments are described below with reference to the accompanying drawings, wherein like numerals denote like elements.

FIGS. 1A-1B provide schematic depictions of an analyte detection system for analyzing the presence, absence, and/or quantity of one or more target analytes within a collected sample, wherein FIG. 1A shows components uncoupled and FIG. 1B shows components coupled for analysis and charging, according to an example embodiment.

FIG. 1C provides a schematic depiction of another analyte detection system for analyzing the presence, absence, and/or quantity of one or more target analytes within a collected sample, wherein a charger is not provided, according to an example embodiment.

FIG. 2 illustrates a perspective view of a sample collection device in a first orientation, according to an example embodiment.

FIG. 3 illustrates a perspective view of the sample collection device of FIG. 2, with a shroud removed.

FIG. 4 illustrates a perspective view of the sample collection device of FIG. 2 in a second orientation.

FIG. 5 illustrates a perspective view of the sample collection device of FIG. 4, with the shroud removed.

FIG. 6 illustrates an exploded view of the sample collection device of FIG. 2.

FIG. 7 illustrates a cross sectional view of the sample collection device of FIG. 2.

FIG. 8A illustrates a partial cross sectional view of the sample collection device of FIG. 2.

FIG. 8B illustrates a partial cross sectional view of the sample collection device of FIG. 2.

FIG. 9 illustrates a perspective view of a collection chamber of the sample collection device of FIG. 2.

FIG. 10 illustrates another perspective view of the collection chamber of FIG. 9.

### DETAILED DESCRIPTION OF CERTAIN EMBODIMENTS

In the following detailed description, reference is made to the accompanying drawings, which form part of the present disclosure. The embodiments described in the drawings and description are intended to be example and not limiting. As used herein, the term “example” means “serving as an example or illustration” and should not necessarily be construed as preferred or advantageous over other embodiments. Other embodiments may be utilized and modifications may be made without departing from the scope of the subject matter presented herein. Aspects of the disclosure, as described and illustrated herein, can be arranged, combined, and designed in a variety of different configurations, all of which are explicitly contemplated and form part of this disclosure.

One aspect of the disclosure is directed to a system for detecting molecules. In various embodiments, the system includes a cartridge device, a reader device removably coupled to the cartridge device, and a sample collection device. According to various embodiments, the sample device is configured to be exposed to a sample for an analysis, such that the sample collection device receives a sample for medical testing. The sample collection device is further configured to be inserted into the cartridge device, which may be inserted into the reader device. The cartridge device and/or the reader device may cause medical testing to be performed on a portion of the sample collected by the sample collection device.

The sample collection device of various embodiments is configured to collect a sample from a specimen. Sample collection devices may be configured to collect cells and other biological material, such as urine, blood, plasma, saliva, etc. One example sample collection device includes a sample collection chamber configured to draw in a first volume of urine and selectively dispense a second volume of urine into an opening in the cartridge device. In other embodiments, the sample collection device may be configured to collect biological material, particulates, or other chemicals from the environment, such as, for example, from a liquid such as tap water, or from a physical surface or other structure.

The sample collection device of various embodiments is sized and shaped to collect a sufficiently large sample from an appropriate location of a specimen and dispense a precise volume of the sample such that it is possible, using the other devices described below, to detect the presence, absence, and/or quantity of one or more target analytes in and/or on the specimen. For example, a sample collection configured to collect urine may be appropriate for collecting target analytes for various tests, including, for example, tests for tracking testosterone levels, drug levels, vitamin levels, and/or fertility. Further, the sample collection device may be appropriate for collecting a target analyte associated with one or more blood diseases, such as HIV. According to various embodiments, a sample collection device is configured to collect a fluid, such as urine, blood, plasma, or saliva, may include features for compressing a wicking portion of the device to expel a precise volume of the sample absorbed on the wicking portion for analyzing the expelled sample.

In various embodiments, a cartridge comprises a housing, which defines an opening and an enclosed space. The cartridge has various features that enable the cartridge to do one or more of the following: receive a sample with target analytes from a sample collection device, store the sample with sample preparation reagents, provide a space for mixing and binding of the target analytes with sample preparation reagents, provide an analysis zone wherein bound target analytes localize over sensors for detection, provide a fluid medium for transporting the bound target analytes to the analysis zone, store and provide a substrate that can undergo a detectable reaction when introduced to the bound target analytes, provide a fluid medium for transporting the substrate to the bound target analytes in the analysis zone, and provide a waste collection zone where waste is stored.

In various embodiments, the cartridge is a substantially closed system wherein the reactions needed to detect the presence, absence, and/or quantity of one or more target analytes occur within the cartridge. The cartridge of such embodiments is said to be “substantially closed” because the only inputs needed into the cartridge system are one or more of the following: a sample from a specimen, energy to facilitate mixing and bound, and a magnetic force to facilitate localization of bound target analytes within an analysis zone; the only outputs from the cartridge are electrical signals. In various embodiments, the cartridge is target-analyte-specific with the included sample preparation reagents selected to detect one or more specific target analytes. Different cartridge types include different reagents intended to identify different target analytes. For example, different cartridge types may include inflammation, influenza, testosterone, fertility, HIV, and Vitamin D which each include application-specific reagents intended to identify different target analytes.

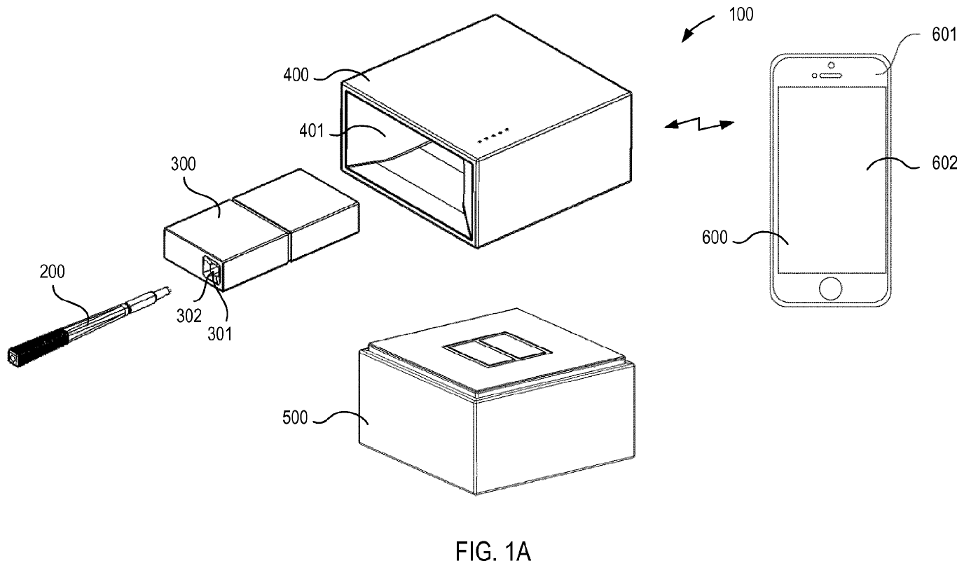

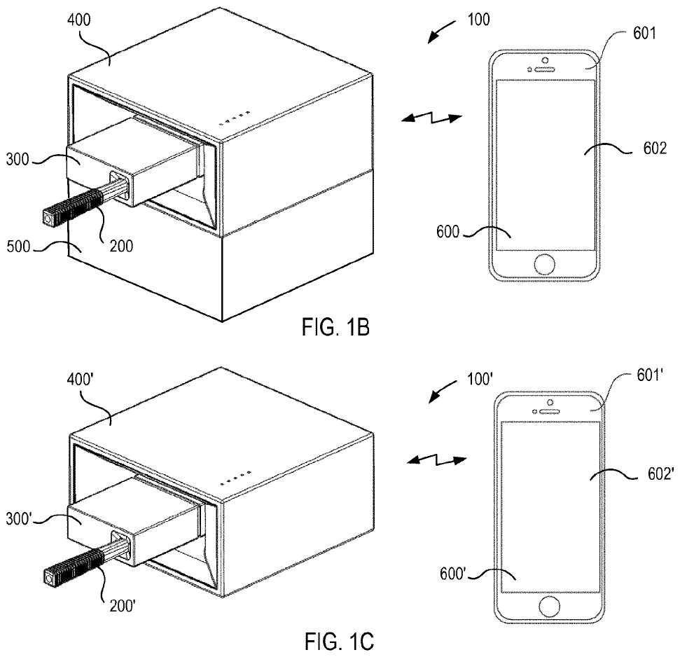

Referring now to FIGS. 1A and 1B, an analyte detection system is shown, according to an example embodiment. The detection system 100 may include a sample a collection device 200, a cartridge device 300, a reader device 400, a charger 500, and/or a software-based detection interface system 600. The detection system 100 may be used to detect the presence, absence, and/or quantity of one or more target analytes.

The sample collection device 200 is configured to be exposed to a sample for analysis. For example, the sample collection device 200 may be exposed to a biological sample, such as, but not limited to, blood, plasma, urine, saliva, mucous, cellular material and/or other biological material for determining the presence, absence, and/or quantity of one or more target analytes within the sample. According to various embodiments, the sample collection device 200 is configured to be submerged in a container holding the sample. For example, a container may be used to collect urine, and a portion of the sample collection device 200 may be submerged in the urine to collect a sample.

The cartridge device 300 is configured to analyze the sample collected with the sample collection device 200. The cartridge device 300 may include an input tunnel 301 that extends from an aperture 302 into the cartridge housing. The input tunnel 301 is configured to permit insertion of the sample collection device 200 as shown in FIG. 1B such that the collected sample may be analyzed within the cartridge device 300. Based on the analysis, the cartridge device 300 is configured to generate electric signals indicative of the presence, absence, and/or quantity of one or more target analytes within the sample.

The reader device 400 is configured for electric coupling with the cartridge device 300 to permit transmission of the electric signals indicative of the presence, absence, and/or quantity of one or more target analytes within the sample generated by the cartridge device 300. The cartridge device 300 may be electrically coupled to the reader device 400 by inserting the cartridge device 300 within the reader opening 401 of reader device 400 as shown in FIG. 1B such that respective electrical connectors of the cartridge device 300 and the reader device 400 contact one another. The reader device 400 may comprise a computer readable medium with instructions that, when executed by a processor of the reader device 400, cause electrical components of the cartridge device 300 to perform steps for analyzing the sample on the sample collection device 200. According to various embodiments, the instructions are not executed until cartridge device 300 is electrically coupled to the reader device 400 and the sample collection device 200 is suitably disposed within the cartridge device 300, for example, as shown in FIG. 1B.

In one embodiment, the sample collection device 200 and the cartridge device 300 are each disposable and designed for one time use while the reader device 400 is designed for multi-use and for receiving many different cartridge devices throughout the life of the reader device 400 such that many samples are analyzed by the reader device 400 for determining the presence, absence, and/or quantity of one or more target analytes within the respective samples. Such a configuration is expected to promote sanitary use of the system, as the components exposed to the sample are disposable, while reducing costs as the components with more expensive electronics, e.g., the reader device 400, may be used repeatedly.

The charger 500 is configured to charge one or more batteries within the reader device 400, e.g., via respective inductive coils disposed within the housings of the charger 500 and the reader device 400. The charger 500 may be plugged into a conventional socket, e.g., via a cord or a cord with an AC to DC power converter, for charging components within the charger 500 to permit charging of the reader device 400.

It should be appreciated that, the detection system need not require a charger. For example, referring to FIG. 1C, a detection system 100′ is constructed similarly to the detection system 100 of FIGS. 1A and 1B, wherein like components are identified by like-primed reference numbers. Thus, for example, a cartridge device 300′ in FIG. 1C corresponds to the cartridge device 300 of FIGS. 1A and 1B, etc. As will be observed by comparing FIGS. 1C and 1B, the detection system 100′ does not include the charger 500. In such an embodiment, the reader device 400′ may be plugged into a conventional socket, e.g., via a cord or a cord with an AC to DC power converter, for powering components of the reader device 400′ and/or the reader device 400′ may include a suitable battery such as a replaceable battery or rechargeable battery and the reader device 400′ may include circuitry for charging the rechargeable battery, and a detachable power cord.

In FIGS. 1A and 1B, a software-based detection interface system 600 is installed and runs on a computing device 601 to permit a user to review analyte detection test results, e.g., on a display 602 of a computing device 601, according to various example embodiments. The computing device 601 may be, for example, a smartphone, smartwatch, tablet, wearable device, a laptop or other computer. As shown in FIGS. 1A and 1B, the reader device 400 may communicate with the computing device 601 wirelessly to transmit data indicative of the presence, absence, and/or quantity of one or more target analytes based on the electrical signals generated within the cartridge device 300. In addition or alternatively, a removable wired connection, such as a cable connection, may be provided between the reader device 400 and the computing device 601. The software-based detection interface system 600 may comprise a computer readable medium with instructions that, when executed by a processor of the computing device 601, cause the display 602 to display information indicative of the presence, absence, and/or quantity of one or more target analytes. It should be appreciated that, according to various embodiments, the software-based detection interface system 600 may be omitted from the detection system 100. For example, the reader device 400 may include a display or other visual indication system that provides results and/or other information to a user of the detection system 100 without the need for an external computing device 601 and/or a software-based detection interface system 600.

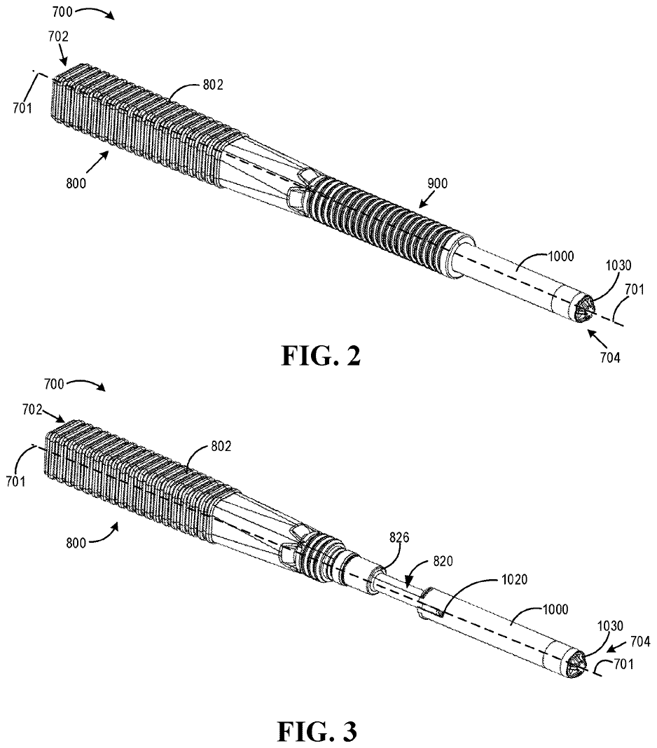

Referring now to FIGS. 2-5, a sample collection device 700 is illustrated, according to an example embodiment. The sample collection device 700 can share one or more features with the other sample collection devices described herein (e.g., the sample collection device 200). For example, the sample collection device 700 can be used in conjunction with the detection system 100 in the same or similar manner as the sample collection device 200 as described above.

The sample collection device 700 is configured to collect a sample and dispense a desired volume of the sample into a desired location to be analyzed. The sample collection device 700 is configured for full or partial insertion within the cartridge device 300 after sample collection. The sample collection device 700 extends along an axis 701 from a first sample collection device end 702 to a second sample collection device end 704. The sample collection device 700 includes a body 800, including a handle portion 802. In use, a user may grasp the sample collection device 700 via the handle portion 802. The handle portion 802 includes a plurality of ridges, which can increase the frictional force between the user's fingers and the handle portion 802 to improve the user's grip.

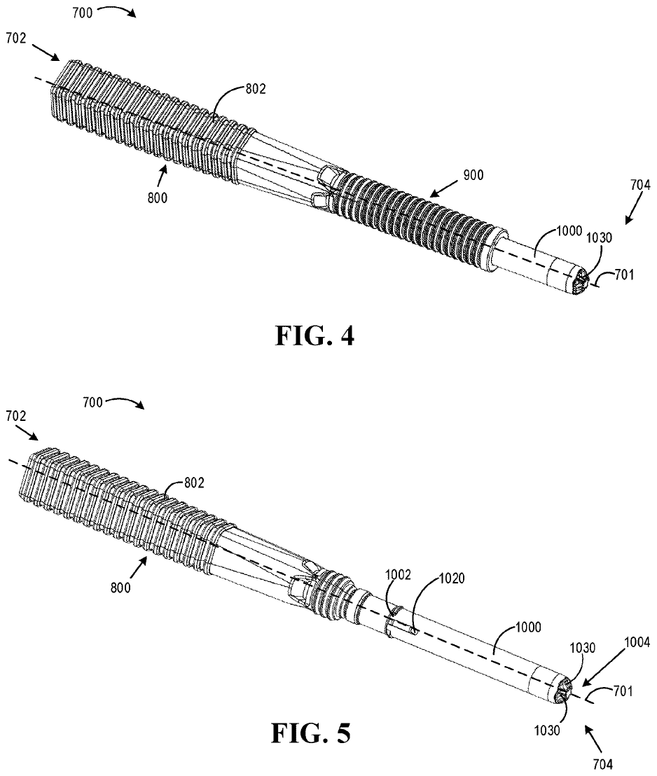

The sample collection device 700 includes a shroud 900 (see FIGS. 2 and 4) coupled to the body 800 between the first sample collection device end 702 and the second sample collection device end 704. As shown in FIGS. 2 and 4, various components are at least partially positioned within an internal volume of the shroud 900. Thus, FIG. 3 and FIG. 5 illustrate the sample collection device 700 with the shroud 900 removed to provide a better understanding of the sample collection device 700.

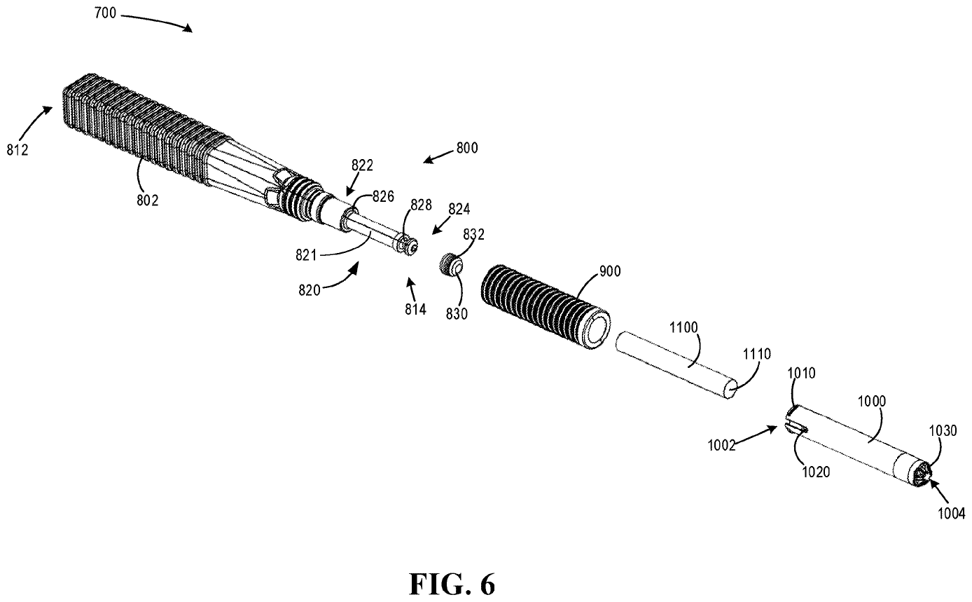

The sample collection device 700 includes a collection chamber 1000 proximate the second sample collection device end 704. The collection chamber 1000 includes an opening 1030 proximate the second sample collection device end 704. When the sample collection device 700 is in a first orientation and the collection chamber 1000 is in a first position (e.g., shown in FIGS. 2 and 3), the opening 1030 is configured to be exposed to a sample such that a minimum desired volume (e.g., a first volume) of the sample is collected within an inner volume of the collection chamber 1000. For example, the sample collection device 700 may include a wick 1100 (see FIG. 6) positioned within the collection chamber 1000. When the opening 1030 is exposed to a sample (e.g., submerged in urine), the wick 1100 functions to absorb a minimum desired volume of the sample via the opening 1030, as is discussed herein. As the sample is absorbed by the wick 1100, air located within the collection chamber 1000 may be released via one or more vents 1020 in the collection chamber 1000.

The sample collection device 700 is further configured to release a desired volume (e.g., a second volume) from the collection chamber 1000 via the opening 1030. The collection chamber 1000 is configured to translate along the axis 701 (e.g., relative to the body 800) between a first orientation (e.g., shown in FIGS. 2 and 3) and a second position (e.g., shown in FIGS. 4 and 5) to release the desired volume of the sample. For example, after a first volume of the sample is collected within the collection chamber 1000 while the collection chamber 1000 is in the first position, an axial force (e.g., along the axis 701) can be applied to the second sample collection device end 704 to cause the collection chamber 1000 to transition from the first position to the second position, thereby putting the sample collection device 700 is in a second orientation. As the collection chamber 1000 transitions to the second position (e.g., shown in FIGS. 4 and 5), a plunger 820 translates into the collection chamber 1000 causing a desired volume (e.g., a second volume) of the sample to be released from the opening 1030, as is described further below. According to various embodiments, the sample collection device 700 is inserted into the aperture 302 in the cartridge device 300 until the second sample collection device end 704 interfaces with a rigid structure within the aperture 302. The user may continue to push the sample collection device 700 into the aperture 302, thereby generating a minimum axial force to act upon the second sample collection device end 704, thereby causing the collection chamber 1000 to translate along the axis 701 from the first position to the second position to dispense a the sample within the aperture 302.

As shown, the body 800 includes a shoulder 826 configured to limit translation of the collection chamber 1000 along the axis 701. For example, when the collection chamber 1000 is in the second position, a first collection chamber end 1002 interfaces with the shoulder 826 to prevent the collection chamber 1000 from translating further towards the first sample collection device end 702. By limiting the distance the collection chamber 1000 translates along the axis 701, the volume of the sample released from the opening 1030 may be controlled. Further, the total volume released from the opening 1030 as the collection chamber 1000 translates from the first position to the second position depends on the distance the collection chamber 1000 translates along the axis 701. Thus, the sample collection device 700 can be configured to dispense a sample volume that is within a predetermined range of volumes (e.g., greater than or equal to a minimum desired volume and less than or equal to a maximum desired volume) by adjusting the distance the collection chamber 1000 travels between the first position and the second position.

As discussed further herein, the collection chamber 1000 may be secured in the first position via one or more tabs (e.g., the projections 1010 shown in FIG. 10) when the sample collection device 700 is removed from its packaging and the shoulder 826 limits the total distance the collection chamber 1000 can travel along the axis 701 such that a predetermined volume of the sample is released from the collection chamber 1000. Dispensing of a predetermined volume of the sample is expected to promote accuracy of analyte analysis as a substantially known quantity of the sample will be analyzed.

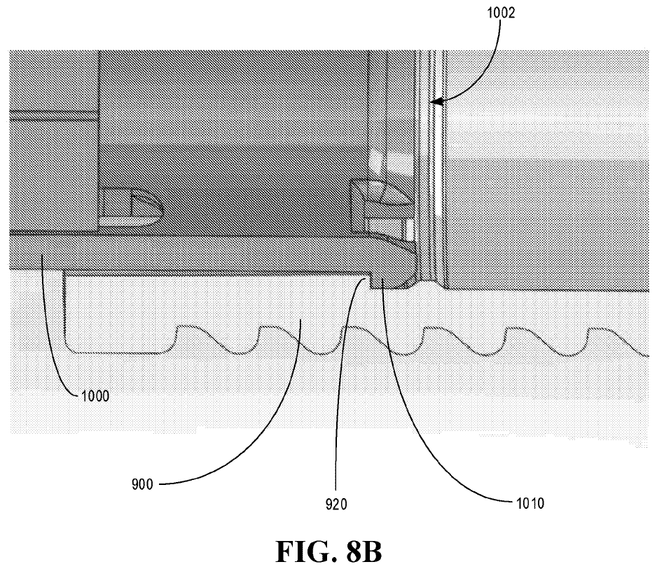

According to various embodiments, at least a portion of the collection chamber 1000 is transparent to permit a collector to verify that wick 1100 is sufficiently saturated. For example, the wick 1100 may include an indicator 1110 (e.g., as shown in FIG. 8A), such as a colored thread, embedded within the wick 1100. As the wick 1100 is saturated, the indicator 1110 within the wick becomes visible via the transparent portion of the collection chamber 1000, which may indicate to the user that a sufficient volume (e.g., a first volume) has been collected.

It should be appreciated that volume of the sample collected by the sample collection device 700 can, in some example embodiments, be greater than the volume of the sample released by the sample collection device 700. For example, the wick 1100 may be configured to absorb a first volume of a sample (e.g., when fully saturated). Since the range of motion of the collection chamber 1000 is limited (e.g., via the shoulder 826), the sample collection device 700 may be configured to dispense a second volume of the sample that is less than the first volume. In other words, the sample collection device 700 may be release all of the sample absorbed by the wick, and excess sample may be discarded along with the sample collection device 700 after use. The sample collection device 700 may be pre-packaged within sterile packaging and may be configured for one-time use.

Referring now to FIG. 6, an exploded view of the sample collection device 700 is shown. The sample collection device 700 includes the body 800, the plunger 820, the shroud 900, the collection chamber 1000, and a wick 1100. While some of the components are shown as separate components, it should be appreciated that one or more of the components may be integrally formed with one another. For example, the body 800 and the plunger 820 are shown as integrally formed components in FIG. 6.

The body 800 extends from a first body end 812 to a second body end 814. As discussed above, the body 800 includes a handle portion 802 proximate the first body end 812. The handle portion 802 is configured to be held by a user during collection of the sample. The body 800 further defines a shoulder 826 proximate the second body end 814. The shoulder 826 defines an outer diameter that is larger than an inner diameter of the collection chamber 1000 such that the shoulder 826 prevents the collection chamber 1000 from translating beyond a desired location relative to the plunger 820.

The plunger 820 includes a projection 821 that extends from the body 800 proximate the second body end 814. For example, the projection 821 is shown to extend from a first plunger end 822 (e.g., proximate the shoulder 826) to a second plunger end 824. The projection 821 defines an elongated body configured to be received within an inner volume of the collection chamber 1000.

The plunger 820 further includes a sealing member 830. As shown, the projection 821 defines a sealing ring 828 proximate the second plunger end 824. The sealing ring 828 is configured to receive the sealing member 830 to couple the sealing member 830 to the projection 821. The sealing member 830 may include a sealing ring 832 configured to interface with the inner walls of the collection chamber 1000 to prevent the sample from flowing past the sealing ring 832 or to reduce the amount of sample that flows past the sealing ring 832. For example, the sealing ring 832 define a diameter that is the same or slightly larger than the inner diameter of the collection chamber 1000 such that the sealing ring 832 compresses within the collection chamber 1000 to create a seal. According to various embodiments, the sealing ring 832 is formed of an elastic material, such as rubber or a polyvinyl.

According to various embodiments, the sealing member 830 is over-molded onto projection 821 proximate the second plunger end 824. For example, the sealing member 830 may be over-molded onto the projection 821 as a part of the manufacturing process. According to various embodiments, due to the relatively small size of the sealing member 830, it may be difficult to couple the sealing member 830 to the projection 821 after both components are formed separately. Therefore, in some example embodiments, over-molding the sealing member 830 onto the end of the projection 821 may reduce manufacturing time and difficulties associated with assembling the sample collection device 700.

Referring still to FIG. 6, the collection chamber 1000 extends from a first collection chamber end 1002 to a second collection chamber end 1004. The collection chamber 1000 includes a projection 1010 proximate the first collection chamber end 1002. As is discussed further below, the projection 1010 interfaces with a projection 920 (see FIG. 8B) of the shroud 900 to prevent the collection chamber 1000 from translating out of the shroud 900. Further, the projection 1010 interfaces with an indentation 910 (see FIG. 8B) of the shroud 900 to restrict undesired movement of the collection chamber 1000 into the shroud, as is discussed further below. For example, a minimum threshold axial force (e.g., about the axis 701) may be required to cause the collection chamber 1000 from translating from the first position towards the second position.

Referring still to FIG. 6, the collection chamber 1000 further includes a vent 1020 proximate the first collection chamber end 1002. The vent 1020 is configured to allow air to escape the collection chamber 1000 as the sample is drawn into the collection chamber via the opening 1030 while the collection chamber 1000 is in the first position. For example, when the collection chamber 1000 is in the first position, the sealing ring 832 may be positioned between the first collection chamber end 1002 and the vent 1020 such that the opening 1030 is in fluid communication with the vent 1020. Since the vent 1020 and the opening 1030 are in fluid communication, the sample can be drawing into the collection chamber 1000 and air can be released from the vent 1020. According to various embodiments, the collection chamber 1000 includes a plurality of vents 1020 surrounding the perimeter of the collection chamber 1000. For example, the collection chamber 1000 may include 2 or more vents equally spaced about the perimeter of the collection chamber 1000.

As the collection chamber 1000 translates towards the second position, the sealing ring 832 passes the vent 1020 and is positioned between the vent 1020 and the second collection chamber end 1004. After the sealing ring 832 passes the vent 1020, the vent 1020 is sealed off from the opening 1030 such that the vent 1020 and the opening 1030 are no longer in fluid communication. As the plunger 820 translates into the collection chamber 1000, the wick 1100 is compressed, thereby forcing at least some of the collected sample out of the opening 1030. Since the sealing ring 832 seals the vent 1020 from wick 1100, the sample is forced out of the opening 1030 and prevented from being released from the vent 1020.

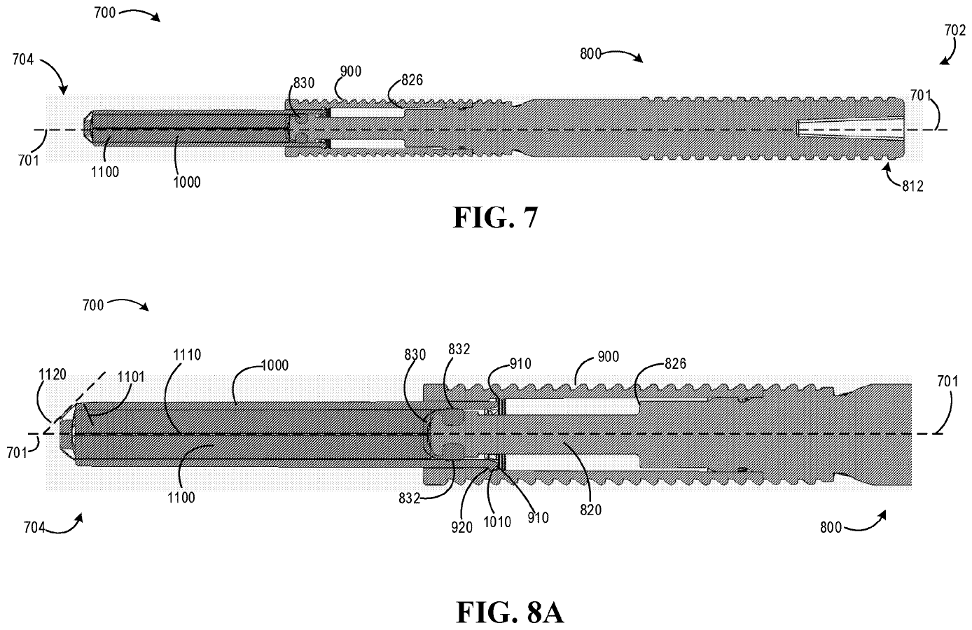

Referring now to FIGS. 7-8B, a cross sectional view and a partial cross sectional views of the sample collection device 700 are shown, respectively. The collection chamber 1000 is shown in the first position in FIGS. 7-8B. Further, the collection chamber 1000 is configured to translate along the axis 701 with respect to the shroud 900 and with respect to the plunger 820 from the first position to a second position. According to various embodiments, the shoulder 826 of the body 800 prevents the collection chamber 1000 from traveling beyond the second position. Since the distance the collection chamber 1000 travels from the first position to the second position is predetermined, the sample collection device 700 is configured to dispense a precise volume of the sample for analysis (e.g., into the aperture 302 of the cartridge device 300.

The collection chamber 1000 includes a projection 1010 that is received within an indentation 910 (e.g., a detent) in the shroud 900 while the collection chamber 1000 is in the first position. The projection 1010 includes a ramped surface, as shown in FIG. 10, that interfaces with the indentation 910 such that the collection chamber 1000 only moves from the first position towards the second position in response to a minimum axial force being applied to the second sample collection device end 704. Because a minimum force is required to cause the projection 1010 to translate out of the indentation 910, the likelihood of accidental release of the sample may be reduced. For example, is the second sample collection device end 704 is subjected to an incidental axial force that is less than the minimum required axial force, the collection chamber 1000 may not translate along the axis 701 towards the second position.

The collection chamber 1000 includes an angled edge 1120 proximate the second sample collection device end 704. The angled edge 1120 form an angle 1101 relative to the axis 701. The angle 1011 may be between less than 90 degrees. For example, the angle 1001 may be less than 60 degrees. For example, the angle 1001 may be approximately 45 degrees. The angled edge 1120 is configured to interface with a corresponding angled face with the aperture 302 of the cartridge device 300, which may facilitate centering of the collection chamber 1000 within the aperture 302. According to various embodiments, centering the collection chamber within the aperture 302 may reduce the risk of compressing the wick 1100 before the second sample collection device end 704 is positioned in the desired location.

The projection 1010 is further configured to interface with a projection 920 inside the shroud 900 to prevent the collection chamber 1000 from translating beyond the first position (e.g., preventing the collection chamber 1000 from being removed from the shroud 900). By controlling the position of the collection chamber 1000 while the collection chamber 1000 is in the first position, the distance between the first position and the second position can also be controlled such that the collection chamber 1000 is configured to dispense a precise volume of a collected sample.

The wick 1100 includes an indicator 1110. According to various embodiments, the wick 1100 may be opaque prior to the sample being collected such that the indicator 1110 (e.g., a colored thread) is not visible or is otherwise difficult to see (e.g., via a transparent portion of the collection chamber 1000). As the wick 1100 draws the sample and becomes at least partially saturated, the indicator 1110 within the wick may become visible. For example, the wick 1100 may become more transparent as the sample is absorbed by the wick 1100 such that the indicator 1110 is visible. Once a user can view the indicator 1110, the wick 1100 may be sufficiently saturated such that a desired volume may be released from the sample collection device 700 (e.g., in response to being inserted into the cartridge device 300).

As shown in FIG. 8B, the collection chamber 1000 includes a projection 1010 (e.g., a shoulder) proximate the first collection chamber end 1002. The projection 1010 interfaces with the projection 920 extending from an interior surface of the shroud 900 to prevent the collection chamber 1000 from translating out of the shroud 900 (e.g., to prevent the collection chamber 1000 from translating beyond the first position).

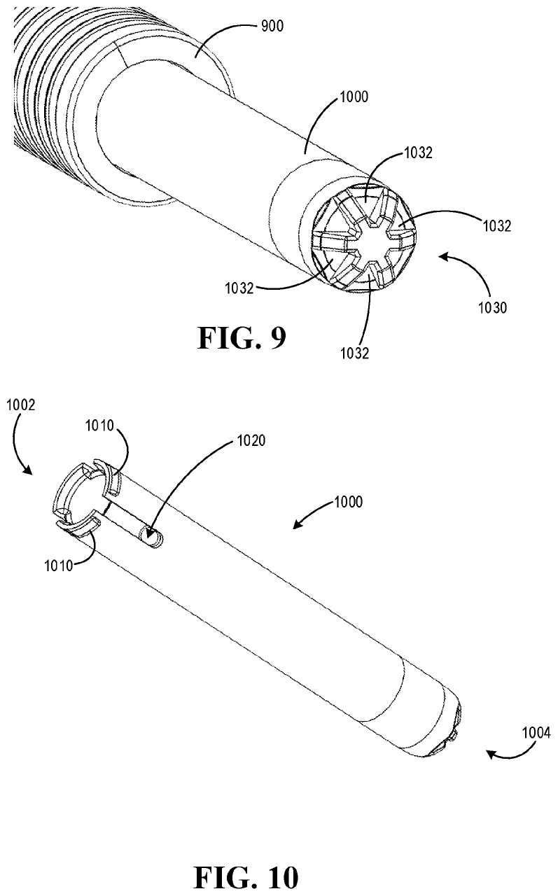

Referring now to FIGS. 9 and 10, perspective views of the collection chamber 1000 are shown. The collection chamber 1000 is configured to collect a sample and release a precise volume of the collected sample for analysis. The collection chamber 1000 extends from a first collection chamber end 1002 to a second collection chamber end 1004. The collection chamber 1000 includes an opening 1030, shown as a plurality of apertures 1032, proximate the second collection chamber end 1004. The plurality of apertures 1032 are equally spaced about a center axis (e.g., the axis 701 shown in FIG. 8A). According to various embodiments, the plurality of apertures 1032 may distribute the sample in a relatively evenly disbursed manner when released from the collection chamber 1000.

Unless otherwise defined, each technical or scientific term used herein has the same meaning as commonly understood by one of ordinary skill in the art to which this disclosure belongs. In accordance with the claims that follow and the disclosure provided herein, the following terms are defined with the following meanings, unless explicitly stated otherwise.

The term “about” or “approximately,” when used before a numerical designation or range (e.g., pressure or dimensions), indicates approximations which may vary by (+) or (−) 5%, 1% or 0.1%.

As used in the specification and claims, the singular form “a”, “an” and “the” include both singular and plural references unless the context clearly dictates otherwise. For example, the term “a molecule” may include, and is contemplated to include, a plurality of molecules. At times, the claims and disclosure may include terms such as “a plurality,” “one or more,” or “at least one;” however, the absence of such terms is not intended to mean, and should not be interpreted to mean, that a plurality is not conceived.

As used in the specification and claims, “at least one of” means including, but not limited to, one or more of any combination of the following. For example, “at least one of A, B, and C” or “at least one of A, B, or C” means including, but not limited to, A(s) or B(s) or C(s) or A(s) and B(s) or A(s) and C(s) or B(s) and C(s) or A(s) and B(s) and C(s); none of which excludes other elements such as D(s), E(s), etc.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a device or method consisting essentially of the elements as defined herein would not exclude other materials or steps that do not materially affect the basic and novel characteristic(s) of the claimed invention. “Consisting of” shall mean that the devices, systems, and methods include the recited elements and exclude anything more than a trivial or inconsequential element or step. Embodiments defined by each of these transitional terms are within the scope of this disclosure.

Although the foregoing has included detailed descriptions of some embodiments by way of illustration and example, it will be readily apparent to those of ordinary skill in the art in light of the teachings of these embodiments that numerous changes and modifications may be made without departing from the spirit or scope of the appended claims.

What is claimed is:

1. A sample collection device for collecting a biological sample for analysis in a detection system, comprising:
a body portion extending along a body axis from a first body end to a second body end;
a collection chamber coupled to the body portion and extending from a first collection chamber end to a second collection chamber end, the collection chamber including an opening proximate the second collection chamber end, the opening configured to receive the biological sample;
a wick positioned within the collection chamber, the wick configured to receive the biological sample from the opening and store a first volume of the biological sample; and
a plunger extending from the body portion proximate the second body end such that a portion of the plunger is positioned within the collection chamber,
wherein the collection chamber is configured to translate relative to the plunger along the body axis between a first position and a second position, and translation of the collection chamber from the first position to the second position causes a second volume of the biological sample to be released from the wick and dispensed from the opening in the collection chamber.

2. The sample collection device of claim 1, wherein the first volume is greater than the second volume.

3. The sample collection device of claim 1, wherein the body portion includes a shoulder proximate the second body end, the shoulder preventing the collection chamber from translating beyond the second position.

4. The sample collection device of claim 1, wherein the collection chamber includes a vent proximate the first collection chamber end, and the opening is in fluid communication with the vent while the collection chamber is in the first position and the opening is not in fluid communication with the vent while the collection chamber is in the second position.

5. The sample collection device of claim 4, wherein the vent is one of a plurality of vents equally spaced about an outer diameter of the collection chamber.

6. The sample collection device of claim 1, wherein the plunger includes a sealing member proximate an end of the plunger, the sealing member including an over-molded portion.

7. The sample collection device of claim 1, further comprising a shroud coupled to the body portion and encompassing at least a portion of the plunger, the shroud including a projection configured to interface with the collection chamber to prevent the collection chamber from translating beyond the first position.

8. The sample collection device of claim 1, wherein the opening is one of a plurality of openings equally spaced apart about the second collection chamber end.

9. The sample collection device of claim 1, further comprising a shroud coupled to the body portion proximate the second body end, the shroud configured to receive a portion of the collection chamber and secure the collection chamber in the second position.

10. The sample collection device of claim 1, wherein the collection chamber includes a transparent portion such that the wick is visible via the transparent portion.

11. The sample collection device of claim 10, wherein the wick is configured to reveal a visual indicator as the wick receives the biological sample saturates the wick.

12. A system, comprising:
a cartridge device comprising a cartridge opening; and
a sample collection device configured to collect a biological sample for analysis and eject the biological sample into the cartridge opening, the sample collection device comprising:
a body portion extending along a body axis from a first body end to a second body end;
a collection chamber configured to be inserted into the cartridge opening and coupled to the body portion and extending from a first collection chamber end to a second collection chamber end, the collection chamber including an opening proximate the second collection chamber end, the opening configured to receive the biological sample;
a wick positioned within the collection chamber, the wick configured to receive the biological sample from the opening and store a first volume of the biological sample; and
a plunger extending from the body portion proximate the second body end such that a portion of the plunger is positioned within the collection chamber,
wherein the collection chamber is configured to translate relative to the plunger along the body axis between a first position and a second position in response to an axial force applied to the body portion while the collection chamber is positioned within the cartridge opening, and
translation of the collection chamber from the first position to the second position causes a second volume of the biological sample to be released from the wick and dispensed into the cartridge opening.

13. The system of claim 12, wherein the first volume is greater than the second volume.

14. The system of claim 12, wherein the body portion includes a shoulder proximate the second body end and the shoulder prevents the collection chamber from translating beyond the second position.

15. The system of claim 12, wherein the collection chamber includes a vent proximate the first collection chamber end, and the opening is in fluid communication with the vent while the collection chamber is in the first position and the opening is not in fluid communication with the vent while the collection chamber is in the second position.

16. The system of claim 12, wherein the plunger includes a sealing member proximate an end of the plunger, the sealing member including an over-molded portion.

17. The system of claim 12, further comprising a shroud coupled to the body portion such that the shroud encompasses at least a portion of the plunger, the shroud including a projection configured to interface with the collection chamber to prevent the collection chamber from translating beyond the first position.

18. The system of claim 12, further comprising a shroud coupled to the body portion proximate the second body end, the shroud configured to receive a portion of the collection chamber and secure the collection chamber in the second position.

19. A sample collection device for collecting a biological sample for analysis in a detection system, comprising:
a collection chamber coupled to a body portion and extending from a first collection chamber end to a second collection chamber end, the collection chamber including an opening proximate the second collection chamber end, the opening configured to receive a first volume of the biological sample; and
a projection extending into the collection chamber, wherein the collection chamber is configured to translate relative to the projection between a first position and a second position, and translation of the collection chamber from the first position to the second position causes a second volume of the biological sample to be released from the collection chamber through the opening.

20. The sample collection device of claim 19, wherein the collection chamber includes a vent proximate the first collection chamber end, and the opening is in fluid communication with the vent while the collection chamber is in the first position and the opening is not in fluid communication with the vent while the collection chamber is in the second position.
